(19) United States
(12) Patent Application Publication
(10) Pub. No.: US 2007/0231320 A1
(43) Pub. Date: Oct. 4, 2007

(54) Eggs having increased antibody titer and methods for making same

(76) Inventors: Mark E. Cook, Madison, WI (US); David L. Trott, Madison, WI (US); Daniel E. Butz, Madison, WI (US)

Correspondence Address: QUARLES & BRADY LLP, 33 E. MAIN ST, SUITE 900, P.O. BOX 2113, MADISON, WI, 53701-2113, US

(21) Appl. No.: 11/396,815

(22) Filed: Apr. 3, 2006

(51) Int. Cl. A61K 39/395; C12P 21/04

(52) U.S. Cl. 424/133.1; 435/70.21

(57) ABSTRACT

A method for increasing antigen-specific antibody titer in an egg by administering a cyclooxygenase inhibitor to an egg-laying animal prior to exposure to the antigen.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND

The invention relates generally to eggs having an increased antibody titer and to methods for making same, and more particularly to methods for increasing egg yolk antibody titer.

In addition to the protective properties conferred by antibodies in vivo in the humoral immune system, antigen-specific antibodies have commercial value, for example, for use as (1) an animal feed supplement, (2) a diagnostic reagent for use in clinical and research laboratory settings and (3) active and passive vaccines. Antibody-containing feed supplements can prevent and can treat infectious disease, can promote growth, can improve feed conversion and can increase yield of animal products such as meat, milk and eggs. Antibodies are advantageous prophylactic and therapeutic alternatives to antibiotics in feed supplements because they do not promote resistance of animal and human pathogens to anti-pathogen drugs, because they do not accumulate in the animal products, and because they are less expensive to develop and produce.

Antibodies produced in egg-laying animals, specifically IgY antibodies, find particular utility in immunological assays. Such antibodies do not (1) cross-react with mammalian IgG, (2) bind to Fc receptors, (3) interact with rheumatoid factors or (4) react with HAMA (human anti-murine antibodies), so non-specific binding is low. Also, secondary antibody-enzyme conjugates made with egg yolk antibodies need not be adsorbed with a mammalian protein to reduce background, as is required for most conjugates that employ mammalian secondary antibody. Conventional chicken egg yolks contain approximately 100-150 mg of IgY immunoglobulin. Unlike yolk, egg albumin contains much lower concentrations of IgY. Each doubling of the antibody titer in an egg reduces the cost of producing an antibody product by 50%.

Methods for producing antibodies, including human monoclonal antibodies, in an egg-laying animal are known to those of skill in the art. Such methods generally include the step of immunizing the animal with an antigen, whereupon serum antibodies to the antigen are accumulated by transporters in the eggs, and particularly in the egg yolks. See Bar-Joseph M & Malkinson M, “Hen egg yolk as a source of antiviral antibodies in the enzyme-linked immunosorbent assay (ELISA): a comparison of two plant viruses,” J. Virol. Methods 1:179-183 (1980); Gassmann M, et al., “Efficient production of chicken egg yolk antibodies against a conserved mammalian protein,” FASEB J. 4:2528-2532 (1990); and Zhu L, et al., “Production of human monoclonal antibody in eggs of chimeric chickens,” Nature Biotechnology 23:1159-1169 (2005), each of which is incorporated herein by reference as if set forth in its entirety.

Methods for advantageously increasing antibody titer in eggs, particularly in egg yolks, have been sought, to further reduce the cost of egg-derived antibodies. Linoleic acid was paradoxically shown to increase antibody titer to one antigen, while simultaneously decreasing antibody titer to a second antigen. See Sijben J, et al., “Immunomodulatory effects of indomethacin and prostaglandin E2 on primary and secondary antibody response in growing layer hens,” Poult. Sci. 79:949-955 (2000) and Sijben J, et al., “Dietary linoleic acid divergently affects immune responsiveness of growing layer hens,” Poult. Sci. 79:1106-1115 (2000).

Sijben et al. hypothesized that these results were caused by prostaglandin E₂ (PGE₂), an eicosanoid known to affect the production of antibodies by B-cells that is synthesized by a cyclooxygenase-dependent pathway, wherein the initial precursor of this pathway is linoleic acid. To test their hypothesis, Sijben et al. injected PGE₂ or indomethacin, a non-selective cyclooxygenase (COX) inhibitor, into poultry just before presenting a first antigen and, in some cases, just before presenting a second antigen. Unfortunately, antibody titers did not increase in a predictable manner for both antigens relative to controls, so the results were inconclusive. The results for one of the antigens, keyhole limpet hemocyanin (KLH), must also be interpreted with caution, as KLH is a known immunostimulant. See McFadden D, et al., “Keyhole limpet hemocyanin, a novel immune stimulant with promising anticancer activity in Barrett's esophageal adenocarcinoma,” Am. J. Surg. 186:552-555 (2003). Furthermore, Sijben et al.'s method, which requires multiple injections and multiple poultry handlings, is impractical and cost-prohibitive when practiced on a large-scale. Therefore, additional methods for consistently producing eggs having a higher egg yolk antibody yield per egg, advantageously with fewer labor requirements, are still sought.

### BRIEF SUMMARY

In one aspect, the invention is summarized in that a method for making an egg having a titer of an antigen-specific egg yolk antibody includes the steps of orally administering to an egg-laying animal a COX inhibitor and then exposing the animal to an immunogenic dose of the antigen, the inhibitor being administered in an amount of sufficient to increase the antibody titer relative to the titer in an animal exposed to the immunogenic dose without prior oral administration of the inhibitor. The animal can be exposed to the antigen at least about thirty minutes, or at least about one hour or about twenty four hours after administration of the COX inhibitor.

In some embodiments, the egg is an avian egg and can be a chicken, duck, emu, goose, ostrich or turkey egg.

In another aspect, the invention is further summarized in that an egg laid by an egg-laying animal treated with an immunogenic dose of an antigen of interest after exposure to a COX inhibitor has an antigen-specific antibody titer at least 50% higher than that in an egg laid by an animal exposed to an immunogenic dose of the antigen without prior exposure to the COX inhibitor.

In some embodiments, the egg-laying animal is an avian animal and can be a chicken, duck, emu, goose, ostrich or turkey.

In some embodiments, the COX inhibitor is indomethacin or aspirin.

In some embodiments, the administered amount of the COX inhibitor is between about 5.5 mg and 110 mg, on average, per animal per day. This can be accomplished using an inhibitor-supplemented animal feed containing at least 50 mg of inhibitor per kg of feed.

### BRIEF DESCRIPTION OF THE DRAWINGS

The invention will be better understood, and features, aspects and advantages other than those set forth above will become apparent when consideration is given to the following detailed description thereof. Such detailed description makes reference to the following drawings, wherein:

FIG. 1 shows the effect over time of orally administered indomethacin on anti-phospholipase A₂ (PLA₂) antibody titer in egg yolk.

FIG. 2 shows the effect over time of orally administered indomethacin and of orally administered aspirin on anti-PLA₂ antibody titer in egg yolk.

FIG. 3 shows the effects of the timing of oral administration of indomethacin and aspirin on anti-PLA₂ antibody titer in egg yolk.

While the invention is susceptible to various modifications and alternative forms, specific embodiments thereof have been shown by way of example in the drawings and are herein described below in detail. It should be understood, however, that the description of specific embodiments is not intended to limit the invention to cover all modifications, equivalents and alternatives falling within the spirit and scope of the invention as defined by the appended claims.

### DETAILED DESCRIPTION OF PREFERRED EMBODIMENTS

Unless defined otherwise, all technical and scientific terms used herein have the same meaning as commonly understood by one of ordinary skill in the art to which the invention belongs. Although any methods and materials similar to or equivalent to those described herein can be used in the practice or testing of the present invention, the preferred methods and materials are described below.

As used herein, a COX inhibitor is defined as any agent that reduces or blocks production of prostaglandin H₂ (PGH₂). The COX inhibitor need not be a selective inhibitor of any COX subtype. Indomethacin (a selective COX-2 inhibitor) and aspirin (a non-selective COX inhibitor) have been determined to be suitable COX inhibitors, but the invention is not so limited. Certain isoforms of conjugated linoleic acid (CLA), such as 10-trans, 12-cis CLA have also been determined to be suitable COX inhibitors. Other suitable COX inhibitors include, but are not limited to, other non-selective COX inhibitors such as ibuprofen, other selective COX inhibitors such as celecoxib, rofecoxib or CLA, anti-COX antibodies and anti-sense molecules to COX.

As used herein, an amount of inhibitor is sufficient if it increases the antigen-specific egg yolk antibody titer by at least about 50%, or at least about 100%, or at least about 200%, or at least about 300% or at least about 400%, relative to the titer obtained in eggs from egg-laying animals exposed to the antigen but not treated with the inhibitor.

The egg-laying animal can be, e.g., an avian animal, a marsupial, a reptile or an amphibian. The animal can be exposed to any antigenic agent against which a humoral immune response can be raised in the animal. The antigen can be a pathogenic agent such as a virus, a bacterium, a fungus, a protozoan, or an antigenic epitope of a pathogenic agent or any other agent against which a humoral response can be raised, such as, but not limited to, a cell surface marker, such as a cancer cell marker. Furthermore, the antigen can be a nucleic acid molecule that encodes an antigen or antigenic determinant, as in published U.S. Patent Publication No. 2004/0087522, incorporated herein by reference as if set forth in its entirety.

The antibody titer in colostrum could also be increased by administering a COX-inhibitor to an animal such as a cow, goat or sheep around the time of exposure to an immunogenic dose of an antigen of interest.

The animal can be exposed to more than one antigen (or more than one epitope) such that more than one antigen-specific antibody is produced and transported to the egg yolk.

A suitable immunogenic dose of antigen is 50-500 μg/ml for a 1 ml injection of an emulsion containing a purified antigen. Alternatively, a suitable immunogenic dose is 3 mg/ml for a 1 ml injection of an emulsion containing an unpurified antigen.

Antibodies can be prepared from the egg yolks using conventional methods available to the skilled artisan. Briefly, yolks can be freeze dried to form a shelf-stable powdered egg yolk product. Yolk antibodies can be purified, e.g., to remove large quantities of lipid. See Camenisch C, et al., “General applicability of chicken egg yolk antibodies: the performance of IgY immunoglobulins raised against the hypoxia-inducible factor 1α,” The FASEB Journal 13:81-88 (1999); Akita E & Nakai S, “Comparison of four purification methods for the production of immunoglobulins from eggs laid by hens immunized with an enterotoxigenic E. coli strain,” J. Immunol. Methods 160:207-214 (1993), each incorporated by reference as if set forth herein in its entirety; as well as incorporated U.S. Pat. Publication No. 2004/0087522. Commercially available egg antibody purification kits, such as EGGstract® IgY Purification Systems (Promega; Madison, Wis.) or Eggcellent® Chicken IgY Purification (Pierce Biotechnology, Inc.; Rockford, Ill.), can also be used to purify the antibodies.

The antibodies themselves can be purified to the required extent and employed in the manner in which antibodies obtained from other sources are used. For example, the antibodies can be used as a feed supplement when mixed with animal feed, or as a passive vaccine when mixed with a pharmaceutically acceptable carrier, or as a diagnostic reagent, especially when provided in a kit with other reagents for a diagnostic assay. Acceptable uses for the antibodies include flow cytometry, Western blotting, immunohistochemistry, latex agglutination and ELISA.

The invention will be more fully understood upon consideration of the following non-limiting Examples.

### EXAMPLES

### Example 1

### Poultry Feed Supplemented with Common COX Inhibitors Increased Antibody Production

Hyline-98 leghorn hens were randomly assigned to treatment groups and given free access to standard breeder's mash and water. In a first set of trials, the hens were divided at 245 days of age into two treatment groups. For twenty four hours after the groups were established, a control group (n=6) was fed standard breeder's mash and a test group (n=6) was fed standard breeder's mash supplemented with 50 mg of indomethacin/ kg diet. After twenty four hours, both groups were injected intramuscularly (i.m.) at four sites (each breast muscle and each leg muscle) with 1 ml total of 3 mg PLA₂/ml of complete Freund's adjuvant (CFA; Sigma, St. Louis, Mo.) and phosphate buffered saline (PBS) in a 50/50 mixture. Hens were given an i.m. booster injection over the four sites seven days after initial injection with 1 ml total of a 3 mg PLA₂/ml of incomplete Freund's adjuvant (IFA; Sigma) and PBS in a 50/50 mixture.

Eggs were cracked and the yolks and albumin were separated. The antibodies were extracted by a 1:10 dilution of yolk in acidified PBS (pH 5.0) for twelve hours. Antibody titer was assayed by enzyme-linked immunosorbent assay (ELISA) at twenty-eight, thirty-five and forty-two days after exposure to the antigen. Optical density was measured at 450 nm using an HRP enzyme reaction to detect antibody. A standard on each plate was defined as a “positive reaction,” which is defined as twice the optical density of an egg yolk diluted 1:2000 from a hen not immunized against the antigen.

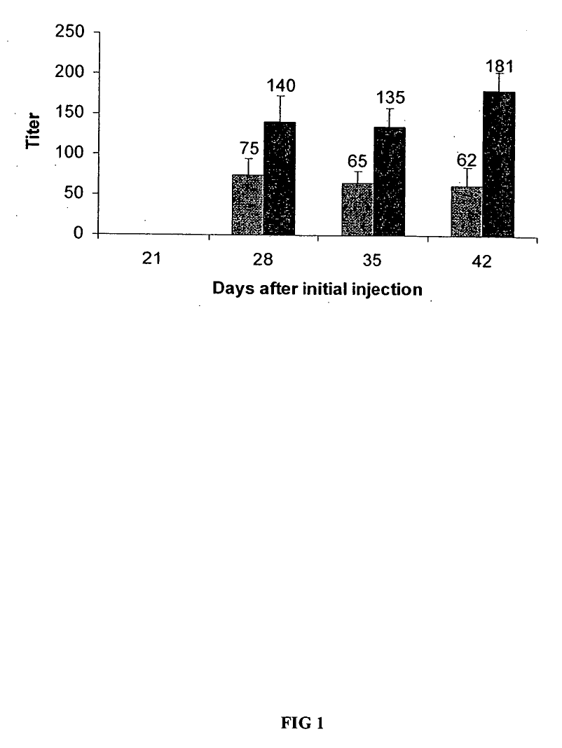

As shown in FIG. 1, by twenty-eight days following exposure to PLA₂, eggs from both control and indomethacin-treated hens contained antigen-specific anti-PLA₂ antibodies. However, the anti-PLA₂ antibody titer was significantly increased in eggs from indomethacin-treated hens at twenty-eight, thirty-five and forty-two days following exposure to PLA₂. At forty-two days, anti-PLA₂ antibody titer of eggs from indomethacin-treated hens was nearly triple the anti-PLA₂ antibody titer of eggs from control hens.

In a second set of trials, the hens were divided at 300 days of age into three treatment groups. For twenty-four hours after the groups were established, a control group (n=6) was fed standard breeder's mash, a first test group (n=6) was fed standard breeder's mash supplemented with 50 mg of indomethacin/kg diet, and a second test group (n=6) was fed standard breeder's mash supplemented with 1 g aspirin/kg diet. After twenty-four hours, the three groups were injected at the four sites with 1 ml total of 3 mg PLA₂/ml of CFA and PBS in a 50/50 mixture. Booster injections were given as described above.

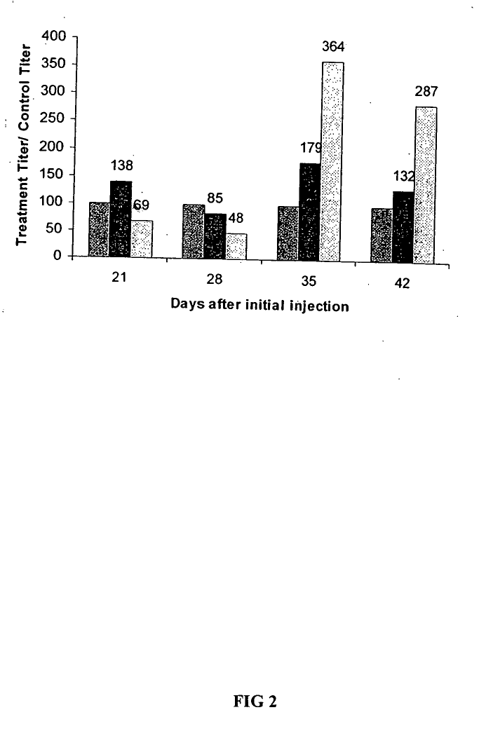

Antibody titer was determined as in the first trial at twenty-one, twenty-eight, thirty-five and forty-two days after exposure to the antigen. FIG. 2 shows that at twenty-one days following exposure to PLA₂, eggs from control, indomethacin-treated, and aspirin-treated hens had anti-PLA₂ antibodies. However, the anti-PLA₂ antibody titer in eggs from both indomethacin- and aspirin-treated hens was significantly increased at thirty-five and forty-two days following exposure to PLA₂. At forty-two days, anti-PLA₂ antibody titer of eggs from aspirin-treated hens was nearly three times that of eggs from control hens. Although both indomethacin and aspirin increased antibody titer, aspirin was more effective at increasing antibody titer, most likely because it is a non-selective COX inhibitor.

In a third set of experiments, the hens were divided at 245 days of age into three treatment groups. On day one, the three groups were injected at the four sites with 1 ml total of 3 mg PLA₂/ml of CFA and PBS in a 50/50 mixture. A control group (n=8) was fed standard breeder's mash for the entire trial. Starting on day fourteen after exposure to the antigen, a first test group (n=8) was fed standard breeder's mash supplemented with 50 mg of indomethacin/kg diet and a second test group (n=8) was fed standard breeder's mash supplemented with 1 g aspirin/kg diet.

Antibody titer was determined as in the first two trials at twenty-one, twenty-eight and sixty-three days after the injection of PLA₂.

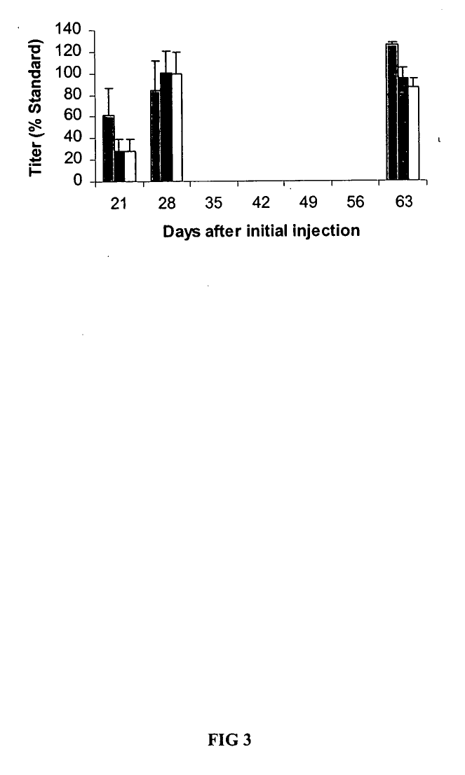

FIG. 3 shows that at twenty-one days following exposure to PLA₂, eggs from control, indomethacin-treated and aspirin-treated hens contained anti-PLA₂ antibodies. The anti-PLA₂ antibody titer, however, was not significantly different in eggs from either treatment group compared to control at twenty-one and twenty-eight days following exposure to PLA₂. In contrast, at sixty-three days following exposure to PLA₂, the control group had a higher antibody titer than either treatment group. Therefore, the time of administering a COX inhibitor is important.

Those of ordinary skill in the art will readily appreciate that the foregoing represents merely certain preferred embodiments of the invention. Various changes and modifications to the procedures and compositions described above can be made without departing from the spirit or scope of the present invention, as set forth in the claims below.

The invention claimed is:

1. A method for making an egg having a yolk that comprises an antigen-specific antibody, the method comprising the steps of:
orally administering a cyclooxygenase inhibitor to an egg-laying animal; and then
exposing the animal to an immunogenic dose of the antigen,
the inhibitor being administered in an amount of sufficient to increase titer of the antigen-specific antibody relative to the titer of antibody specific to the antigen in an animal exposed to the immunogenic dose without the inhibitor.

2. A method as claimed in claim 1, wherein the antigen is administered at least about 30 minutes after administration of the cyclooxygenase inhibitor.

3. A method as claimed in claim 1, wherein the antigen is administered at least about one hour after administration of the cyclooxygenase inhibitor.

4. A method as claimed in claim 1, wherein the antigen is administered about 24 hours after administration of the cyclooxygenase inhibitor.

5. A method as claimed in claim 1, wherein the egg-laying animal is selected from the group consisting of an avian, a marsupial, a reptile and an amphibian.

6. A method as claimed in claim 5, wherein the avian animal is selected from the group consisting of a chicken, a duck, an emu, a goose, an ostrich and a turkey.

7. A method as claimed in claim 5, wherein the avian animal is a chicken.

8. A method as claimed in claim 1, wherein the cyclooxygenase inhibitor is selected from the group consisting of indomethacin and aspirin.

9. A method as claimed in claim 1, wherein the cyclooxygenase inhibitor is aspirin.

10. An egg having an antigen-specific egg yolk antibody titer of at least 15-17 mg/ml.

11. An egg as claimed in claim 10, wherein the egg is an avian egg.

12. An egg as claimed in claim 11, wherein the avian is selected from the group consisting of a chicken, a duck, an emu, a goose, an ostrich and a turkey.

13. An egg as claimed in claim 11, wherein the avian is a chicken.

14. An egg as claimed in claim 10, wherein the egg is produced according to a method comprising the steps of orally administering a cyclooxygenase inhibitor to an egg-laying animal and then exposing the animal to an immunogenic dose of the antigen, the inhibitor being administered in an amount of sufficient to increase titer of the antigen-specific antibody relative to the titer of antibody specific to the antigen in an animal exposed to the immunogenic dose without the inhibitor.

15. An egg as claimed in claim 14, wherein the antigen-specific egg yolk antibody titer is at least 50% higher than that of an egg produced without administering the inhibitor to the animal.
